(19) United States
(12) Patent Application Publication
(10) Pub. No.: US 2006/0122640 A1
(43) Pub. Date: Jun. 8, 2006

(54) Surgical tool arrangement

(76) Inventors: Jeremy R. Gordon, Paw Paw, MI (US); Megan S. James, Portage, MI (US)

Correspondence Address: FLYNN, THIEL, BOUTELL & TANIS, P.C., 2026 RAMBLING ROAD, KALAMAZOO, MI, 49008-1631, US

(21) Appl. No.: 11/002,691

(22) Filed: Dec. 2, 2004

(51) Int. Cl. A61B 17/32

(52) U.S. Cl. 606/180

(57) ABSTRACT

A tool arrangement including a powered surgical handpiece and an attachment for use therewith which supports a surgical cutting accessory. The attachment includes an arrangement which automatically axially positions the cutting accessory relative to the attachment upon coupling the attachment to the handpiece. A sleeve arrangement is also provided in the attachment which protects bearings, which are provided for rotatably supporting the cutting accessory, from contamination.

### FIELD OF THE INVENTION

This invention generally relates to a surgical tool arrangement, and more particularly to a surgical tool arrangement including a powered handpiece and an improved attachment usable therewith which supports and accurately positions a surgical cutting accessory driven by the handpiece.

### BACKGROUND OF THE INVENTION

Powered surgical tools are utilized extensively in modern surgery. Typically, this tool includes the handpiece which houses a motor. A cutting accessory is secured to the handpiece for accomplishing a specific medical task. More specifically, some powered surgical tools are provided with drills or burrs for cutting bores into bone or hard tissue or for selectively removing portions thereof. Still other powered surgical tools are provided with cutting accessories such as saw blades for separating large sections of hard tissue. The ability to use powered surgical tools has lessened the physical strain on physicians and other personnel when performing medical procedures on a patient. Moreover, most surgical procedures can be performed more quickly and more accurately with powered surgical tools than with the manual equivalents that preceded them.

Handpieces of the type described above are often utilized with complementary attachments which define the distal head of the handpiece. These attachments function in various ways depending upon the mode in which the handpiece is to be used. For example, some attachments include linkages which transfer the motive power from the handpiece motor to the cutting accessory. Other attachments position the cutting accessory at an angle relative to the longitudinal axis of the handpiece to provide the surgeon with alternative access to the surgical site. Further, some attachments translate the rotary motion of the handpiece rotor into a reciprocating motion, for example when a saw blade is to be used with the handpiece as the cutting accessory. Still other attachments function to provide physical support for a cutting accessory which rotates along with the handpiece rotor, to prevent the shaft of the cutting accessory from bending when exposed to significant loads during surgery. The instant invention generally relates to this latter type of attachment.

In some instances, it is necessary to cut through and remove parts of a skull of a patient, and in this situation a special attachment, typically referred to as a duraguard, is often utilized with a powered handpiece which assists in this procedure and also protects the dura mater or the dura (the thick and fibrous membrane that lines the interior of the skull) from the tip of the cutting accessory during cutting of the skull. This type of attachment incorporates a distally-located foot which is oriented transversely relative to the longitudinal axis of the handpiece and attachment so as to extend over the free distal end of the cutting accessory which projects from the attachment. The foot is typically utilized by the surgeon to peel the dura away from the skull, and at the same time shields the dura from the moving end of the cutting accessory.

When a powered handpiece is utilized with an attachment of the above type, it is important that the cutting accessory be correctly axially positioned relative to the attachment foot. More specifically, the terminal distal or working end of the cutting accessory should be spaced proximally or rearwardly from the foot so as to avoid contact therewith and allow the shaft of the cutting accessory to spin freely. In this regard, if the cutting tip is positioned too far in the forward or distal direction so that the cutting tip makes contact with the foot, the cutting tip could cause fragmenting or separation of the foot from the attachment and/or could cause the foot to become heated due to friction with the tip, either of which is non-desirable. Oppositely, if the cutting tip is positioned too far from the foot in the rearward or proximal direction, then the cutting accessory may not cut through the entire depth or thickness of the skull, resulting in an inadequate cut.

In order to address the above problems, a known duraguard attachment manufactured by the instant assignee incorporates therein a ball-detent arrangement for axially positioning the cutting accessory. This arrangement includes a cutting accessory defining therein a concave annular groove which cooperates with balls located in the attachment in surrounding relation with the cutting accessory. A collar is provided on the attachment which is manipulated by the user to load and unload the cutting accessory. With this attachment, the collar is set to the “run” position, and the cutting accessory is inserted into the attachment until the balls seat within or “find” the accessory groove. The attachment and cutting accessory are then installed on the handpiece by manipulating a collar provided on the handpiece. The attachment is removed from the handpiece by again manipulating the handpiece collar, and then the cutting accessory is removed from the attachment by setting the attachment collar to the “unlock” position. A disadvantage of this arrangement is that the user must manually manipulate two collars during assembly. That is, one collar must be used to lock the attachment to the handpiece, and another collar must be used to position the cutting accessory relative to the attachment.

The present invention prevents or at least minimizes the above shortcomings of the known arrangement, in that the attachment and cutting accessory according to the invention cooperate to automatically set the axial position or depth of the cutting accessory relative to the attachment, without the need for a separate collar on the attachment. More specifically, the user simply inserts the cutting accessory either into the attachment or alternatively inserts the cutting accessory directly into the distal end of the handpiece, and then secures the attachment to the handpiece. The attachment incorporates therein a positioning arrangement which engages the accessory shaft and cooperates therewith to maintain the shaft in a predetermined axial position relative to the attachment when same is coupled to the handpiece. The arrangement according to the invention correctly sets the depth of the cutting accessory relative to the foot of the attachment, without the surgeon having to manually manipulate a separate collar.

In addition, the attachment according to the invention incorporates therein an improved bearing arrangement, wherein the bearings within the attachment which support the cutting accessory are shielded by a sleeve-like component interposed between the shaft of the cutting accessory and the bearings. In the arrangement disclosed in U.S. Pat. No. 5,888,200 owned by the assignee hereof, the bearings located in the attachment are in direct contact with the cutting accessory. This known arrangement allows saline and other surgical debris to make contact with the bearings, which can effectively shorten the life thereof. The improved bearing arrangement according to the invention, however, routes fluid and other surgical debris through the attachment and away from the bearings, which results in longer-life bearings.

### BRIEF DESCRIPTION OF THE DRAWINGS

FIG. 1 is a perspective view of the surgical tool arrangement according to the invention;

FIG. 2 is a partially exploded perspective view of the surgical tool arrangement of FIG. 1;

FIG. 3 is an enlarged, longitudinal cross-sectional and fragmentary view of the distal end of the surgical tool arrangement illustrating same in an assembled tool “run” state;

FIG. 4 is an enlarged, longitudinal cross-sectional view of the attachment;

FIG. 5 is view similar to FIG. 4, but showing the cutting accessory positioned within the attachment;

FIG. 6 is an enlarged front view of the foot as seen along line 6-6 in FIG. 5;

FIG. 7 is an enlarged and fragmentary side view of the arm and foot of FIG. 5;

FIG. 8 is an exploded perspective view of the attachment of FIG. 4;

FIG. 9 is an enlarged side view of the cutting accessory;

FIG. 10 is an enlarged, cross-sectional view of the cutting accessory of FIG. 9, taken substantially along line 10-10 in FIG. 9;

FIG. 11 is an enlarged view of the distal end of the cutting accessory of FIG. 9;

FIG. 12 is an enlarged, cross-sectional view of a second embodiment of an attachment which may be utilized in the surgical tool arrangement according to the invention;

FIG. 13 is an exploded perspective view of the attachment of FIG. 12;

FIG. 14 is an enlarged fragmentary view of the foot of the attachment in a use position;

FIG. 15 is an enlarged, cross-sectional view of a third embodiment of an attachment which may be utilized in the surgical tool arrangement according to the invention;

FIG. 16 is an exploded perspective view of the attachment of FIG. 15;

FIG. 17 is an enlarged and fragmentary perspective view of a second embodiment of a cutting accessory which may be utilized in the surgical tool arrangement according to the invention;

FIG. 18 is an enlarged and fragmentary perspective view of a third embodiment of a cutting accessory which may be utilized in the surgical tool arrangement according to the invention; and

FIG. 19 is an enlarged and fragmentary perspective view of a fourth embodiment of a cutting accessory which may be utilized in the surgical tool arrangement according to the invention.

Certain terminology will be used in the following description for convenience in reference only, and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center of the tool arrangement and designated parts thereof. The words “forwardly” and “distally” will refer to the direction toward the end of the tool arrangement which is closest to the patient, and the words “rearwardly” and “proximally” will refer to the direction away from the end of the tool arrangement which is furthest from the patient. Said terminology will include the words specifically mentioned, derivatives thereof, and words of similar import.

### DETAILED DESCRIPTION

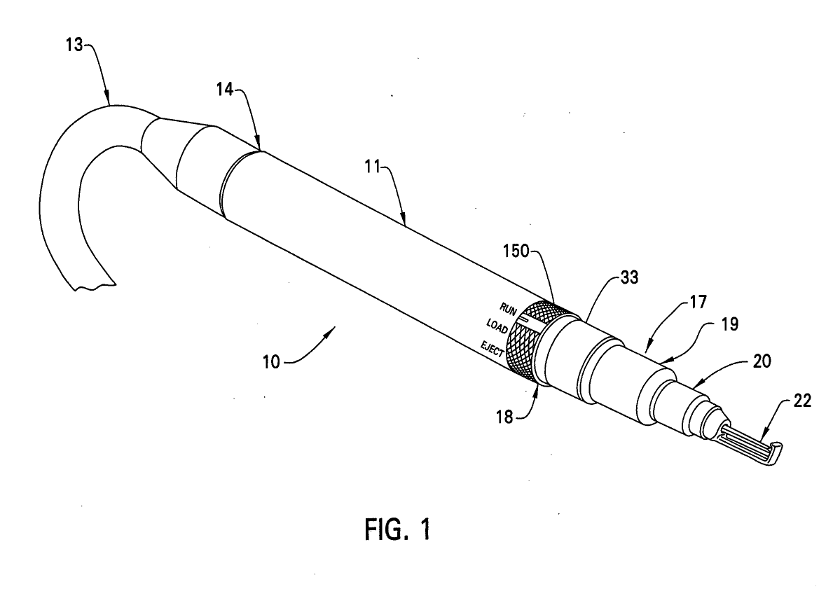

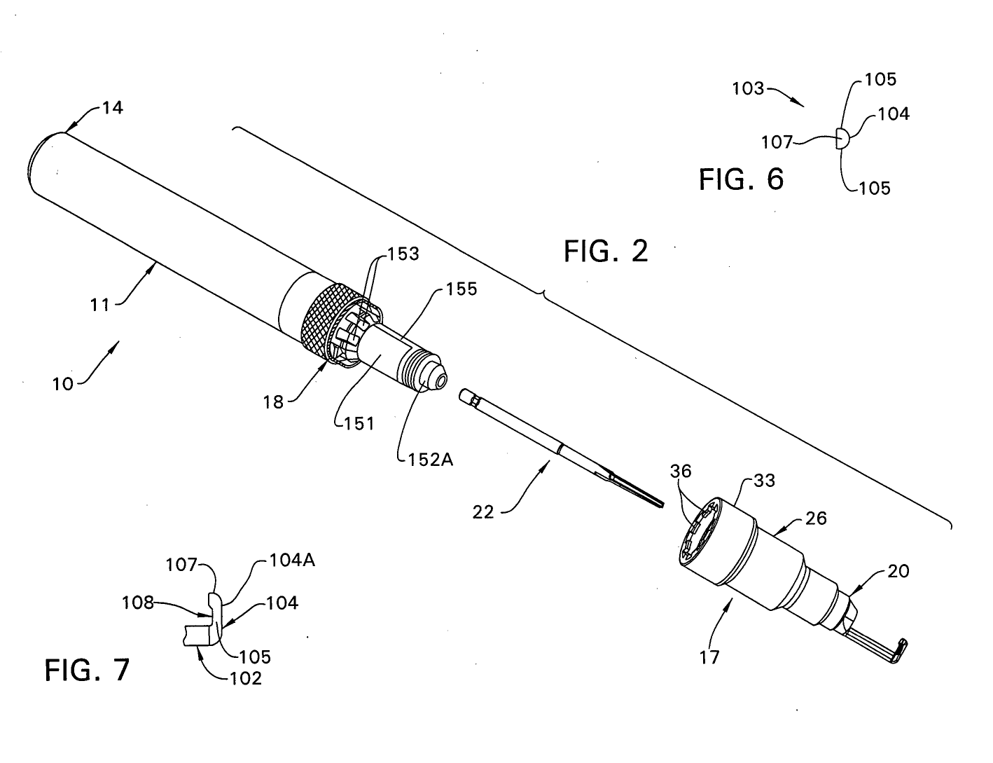

FIGS. 1-3 illustrate the surgical tool arrangement 10 according to the invention. Arrangement 10 includes the handpiece 11 in which a motor is housed. In the illustrated embodiment, handpiece 11 includes an electric motor 12 having a rotor or rotor shaft 12A, although the handpiece 11 may instead include a pneumatic motor, or indeed any other convenient type of motive power source. Thus, the term “rotor” as used herein in association with the handpiece motor is not to be considered to be limited solely to a rotor or rotating element of an electric motor. The motor 12 is energized with power supplied to the handpiece 11 via a cable 13 coupled to a proximal or base end 14 of the handpiece 11.

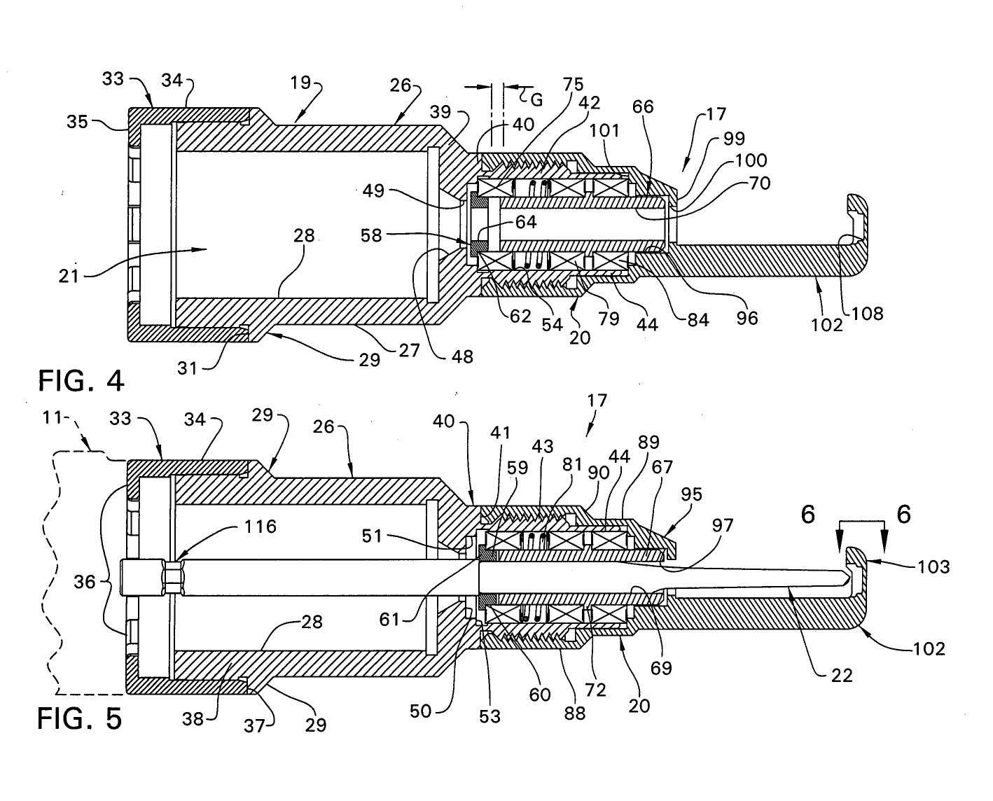

The arrangement 10 additionally includes an attachment 17 which is coupled to a distal or head end 18 of the handpiece 11. Attachment 17 includes a housing 19 which is physically coupled to the handpiece 11, and a cap or guard 20 coupled to housing 19 and defining the distal end of the arrangement 10. Housing 19 and cap 20 together define a bore 21 (FIG. 4) which extends throughout the entire longitudinal extent of the attachment 17 and during operation is occupied by a cutting accessory 22. In the illustrated embodiment, the cutting accessory 22 is a router which is coupled to rotor 12A of motor 12 of handpiece 11. It will be appreciated that other cutting accessories may be utilized, and that accessory/router 22 is only an example of one type of accessory which may be utilized in accordance with the invention.

The structure and operation of handpiece 11 illustrated in FIGS. 1-3 and its cooperation with a conventional attachment is disclosed in detail in U.S. Pat. No. 5,888,200 (herein “the '200 patent”), which is incorporated by reference herein in its entirety. Accordingly, the structure and functioning of the handpiece 11 will be only briefly described herein in relation to the attachment 17, following the description of the attachment 17.

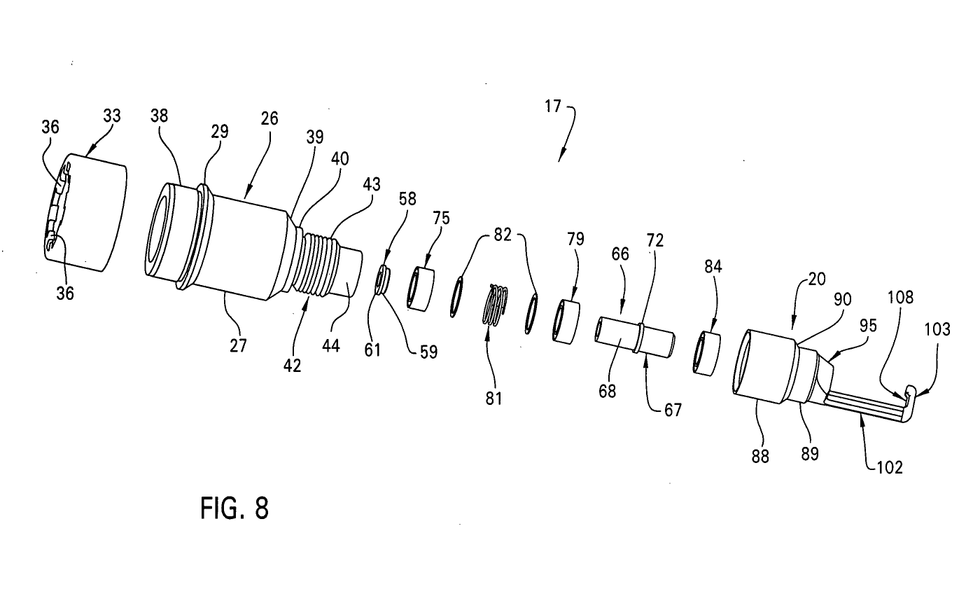

With reference to FIGS. 4, 5 and 8, housing 19 of attachment 17 is defined by a generally cylindrical base 26. Base 26 includes a cylindrical part 27 which defines a bore 28 of a constant diameter. Cylindrical part 27 defines thereon an outwardly projecting annular flange 29, which is tapered on its distal side and at its proximal side defines a shoulder 31 which is oriented generally perpendicularly relative to the outer surface of part 27.

A retainer ring 33 is fixed to the proximal end of base 26. Retainer ring 33 is defined by a cylindrical sleeve 34 and an annular lip 35 which projects inwardly from a proximal end of sleeve 34 and is generally perpendicular thereto. Lip 35 defines a plurality of inwardly projecting teeth 36 which are spaced uniformly and circumferentially about lip 35. As shown in FIGS. 4 and 5, retainer ring 33 is fixed to the proximal end of base 26. More specifically, ring 33 is disposed in surrounding relationship with an end 38 of cylindrical part 27 located proximally of flange 29, and a terminal distal end face 37 of ring 33 is seated against shoulder 31 of part 27.

With continued reference to FIGS. 4, 5 and 8, cylindrical part 27 includes an angled section 39 which tapers inwardly as same projects distally and adjoins a cylindrical neck 40. Neck 40 defines thereon a distally facing annular shoulder 41 which adjoins an elongate sleeve-shaped portion 42 of a reduced outer diameter than neck 40. Sleeve-shaped portion 42, adjacent neck 40, defines thereon an externally threaded section 43, and terminates at a generally cylindrically-shaped distal end section 44.

Tapered section 39 of base 26 includes an inner surface which defines a frustoconically-shaped first bore 48 and a cylindrically-shaped second bore 49. Bore 48 is located at the distal end of and communicates with bore 28 of cylindrical part 27, and bore 49 is located distally of and communicates with bore 48. Tapered section 39 also defines an inner and forwardly facing annular surface 50 which adjoins in a perpendicular manner an inner annular surface 51 of neck 40. Neck 40 additionally defines an annular shoulder 53 which faces distally and generally perpendicularly adjoins annular surface 51. Sleeve-shaped portion 42 of base 26 defines therein a longitudinal bore 54 of a constant diameter. Bore 54 is located distally of and communicates with bores 48, 49 and the space defined by surface 51 of neck 40.

Attachment 17 additionally includes a rear bearing sleeve 58 which is disposed in bore 54 of sleeve-shaped portion 42. Rear bearing sleeve 58 is defined by a cylindrical wall 59 having an outer surface 60, and a flange 61 which projects outwardly from the proximal end of wall 59 and is generally perpendicular relative to outer surface 60 thereof. A distally-facing surface of flange 61 perpendicularly joins outer surface 60 and defines a shoulder 62 of rear bearing sleeve 58. Rear bearing sleeve 58 defines therein a bore 64 which extends completely therethrough.

An elongated front bearing sleeve 66 is also disposed in bore 54 of sleeve-shaped portion 42, forwardly of rear bearing sleeve 58. Front bearing sleeve 66 is defined by a cylindrical wall 67 having outer and inner generally parallel surfaces 68 and 69. Inner surface 69 defines a bore 70 which extends completely through sleeve 66 and has a constant diameter therealong which is similar in dimension to that of bore 64 of rear bearing sleeve 58. As shown in FIGS. 4, 5 and 8, front bearing sleeve 66 has an annular spacer 72 which projects outwardly and perpendicularly from outer surface 68, part-way between the proximal and distal ends of sleeve 66.

A first annular ball bearing 75 is disposed within bore 54 of sleeve-shaped portion 42 of base 26, and radially between sleeve-shaped portion 42 and front and rear bearing sleeves 66 and 58. In the illustrated embodiment, bearing 75 and rear bearing sleeve 58 are press-fit together so as to define an integrated bearing for ease of assembly, while the inner diameter of bearing 75 slip-fits over the proximal end of front bearing sleeve 68.

A second annular ball bearing 79 substantially identical to ball bearing 75 is disposed within bore 54, radially between sleeve shaped portion 42 and front bearing sleeve 68. Second ball bearing 79 is axially spaced from first ball bearing 75 by a biasing member, which in the illustrated embodiment is a coil spring 81. A pair of washers 82 are provided at the respective ends of the bearings 75 and 79 which face one another, and spring 81 is disposed between the respective washers 82. In the illustrated embodiment, the inner diameter of bearing 79 slip-fits over the front bearing sleeve 68.

A third annular ball bearing 84 substantially identical to bearings 75 and 79 is located forwardly of second ball bearing 79, radially between the distal end of sleeve-shaped portion 42 and front bearing sleeve 66. Third bearing 84 is separated from second bearing 79 by spacer 72 of front bearing sleeve 66. Bearing 84, in the illustrated embodiment, is press-fit over the distal end of front bearing sleeve 68.

With continued reference to FIGS. 4, 5 and 8, cap 20 of attachment 17 will now be described. In the illustrated embodiment, cap 20 is configured as a guard which is usable to separate the dura from the skull and also to protect the dura during a cutting procedure.

Cap 20 includes a generally cylindrically-shaped annular sleeve-like portion 88 which defines the proximal terminal end of cap 20. Sleeve-like portion 88 defines internal threads which are engageable with the threads of section 43 of base 26 to secure cap 20 on base 26. Sleeve-like portion 88 is joined to a further sleeve part 89 by a transitional section 90 which tapers inwardly as same projects distally. Transitional section 90 and sleeve part 89 together define a cylindrical bore of a constant diameter in which the distal end section 44 of sleeve-shaped portion 42 is disposed.

Cap 20 additionally includes a nose 95 having an inner cylindrically-shaped surface 96 which defines a bore 97 of a constant diameter, and in which the terminal front end of front bearing sleeve 66 is disposed. Nose 95 defines thereon a lip 99 which projects inwardly and perpendicularly relative to inner surface 96 and terminates at an edge which defines a bore 100 which opens outwardly. Bore 100 communicates with bore 97 and is of a reduced diameter as compared to bore 97. Nose 95 additionally defines a shoulder 101 which is generally parallel to and spaced forwardly from shoulder 53 of neck 40.

With reference to FIGS. 4-7, an elongate arm 102 projects distally from lip 99 of nose 95, and at its most distal end is fixed to a foot 103 which is generally perpendicularly oriented relative to arm 102. Foot 103 has an outer wall 104 which has a generally flat exterior surface 104A defining the distal-most extent of cap 20, a pair of side walls 105 which adjoin outer wall 104 through respective rounded edge surfaces, and an end wall 107 which adjoins outer and side walls 104 and 105 through respective rounded edge surfaces. Outer, side and end walls 104, 105 and 107 of foot 103 together define a pocket or recess 108 which opens proximally.

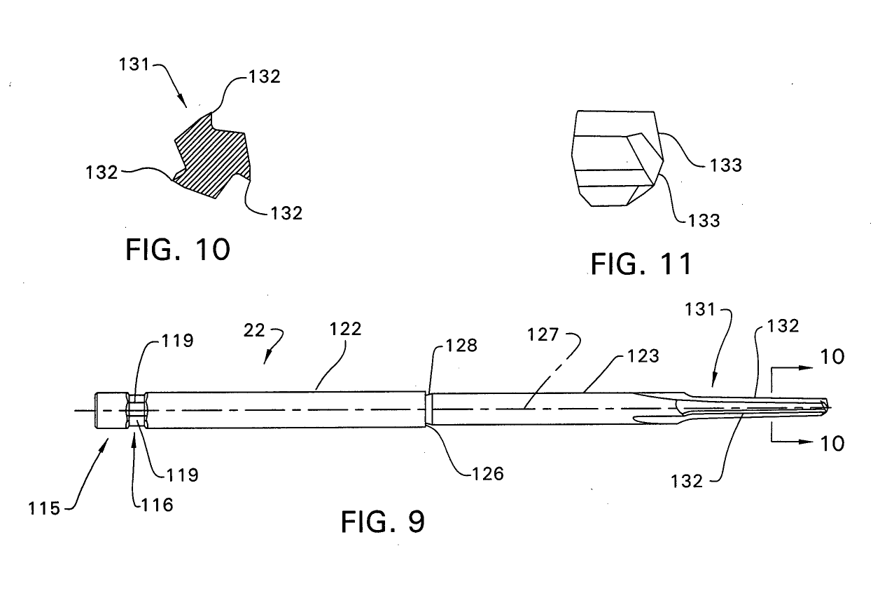

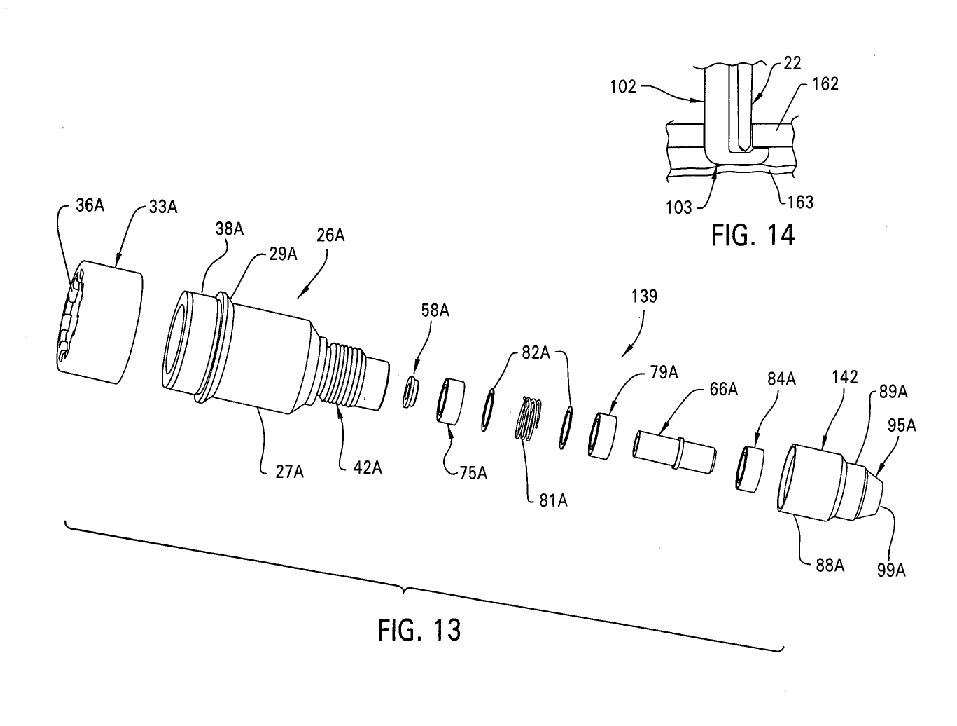

Turning now to cutting accessory 22, and with reference to FIGS. 9-11, same includes a terminal proximal or butt end 115 which is normally seated within the handpiece 11 and rotates along with rotor 12A of motor 12 as discussed below. Cutting accessory 22 includes a shaft-locking section 116 disposed distally of butt end 115. The shaft locking section 116 is identical to the shaft-locking section illustrated in FIGS. 13A and 13B of the '200 patent and cooperates with the handpiece 11 in the same manner as described therein. Thus, this portion of the accessory 22 will be only briefly described.

Shaft-locking section 116 defines thereon a plurality of flat faces 119 which are recessed inwardly relative to the outer diameter of the adjacent butt end 115 and the remainder of the cutting accessory 22. These faces 119 interact with a locking mechanism of handpiece 11 as discussed below which transfers the rotational motion of rotor 12A associated with motor 12 to the accessory 22.

Cutting accessory 22 additionally includes a solid cylindrical portion 122 which extends distally from locking section 116 and has approximately the same diameter as butt end 115. A solid cylindrical portion 123 is located distally of cylindrical portion 122. Portion 123 is formed to have a slightly lesser diameter than portion 122, such that a distally facing shoulder 126 is defined therebetween. Shoulder 126 defines the terminal distal end of portion 122, and has a surface which is perpendicular to a longitudinal axis of 127 of accessory 22. In the illustrated embodiment, for ease of manufacture, accessory 22 includes a transitional section 128 which tapers outwardly as same projects distally away from shoulder 126. However, it will be appreciated that shoulder 126 may instead be formed by forming an annular and circumferentially-extending groove between portions 122 and 123, wherein the groove has a bottom parallel to the longitudinal axis 127 of accessory 22.

As shown in FIGS. 9 and 10, accessory 22 includes cutting tip 131 extending distally from cylindrical portion 123. Tip 131 defines thereon a plurality of longitudinally extending cutting edges 132, which, when the accessory 22 undergoes a sideward motion transverse to the longitudinal axis 127, allows the accessory to be used as a mill. Tip 131 also includes a plurality of cutting edges 133 located at the terminal distal end thereof, as best shown in FIG. 11. These cutting edges 133 allow the accessory 22 to be used as a boring tool or drill when the accessory 22 is advanced toward the bone or hard tissue in a direction along the longitudinal axis 127, for example when accessory 22 is used with attachments other than attachment 17 which are open or non-obstructed at the distal free end thereof.

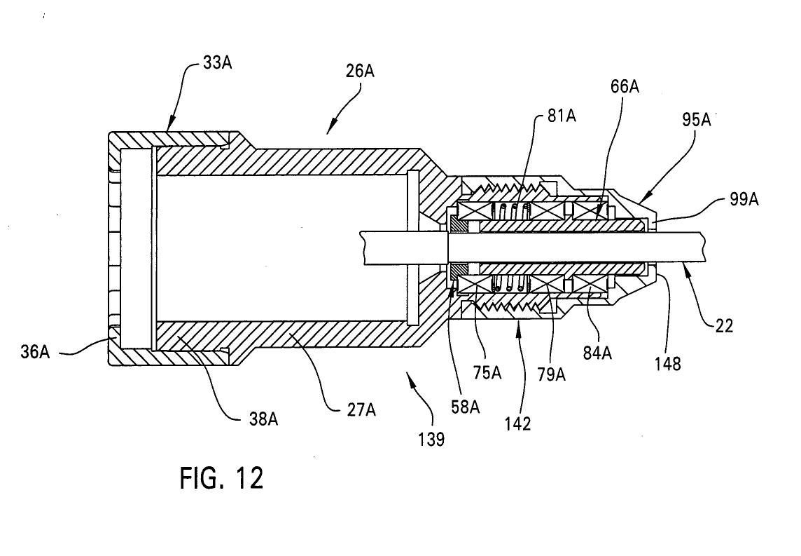

FIGS. 12 and 13 illustrate a second embodiment of an attachment which is usable in the surgical tool arrangement according to the invention. More specifically, these figures illustrate an attachment 139 which is identical to attachment 17, except for the configuration of the cap. Accordingly, components of attachment 139 which are similar or identical components of attachment 17 are identified in FIGS. 12 and 13 with the same references numbers, plus an “A”.

Cap 142 of attachment 139 is identical to cap 20 of attachment 17, except that the nose 95A thereof does not include an arm (such as arm 102) cantilevered outwardly therefrom. Instead, flange 99A of nose 95A defines a generally flat or planar distally-facing end surface 148.

The assembly of a conventional attachment to handpiece 11 is described in detail in the '200 patent, and attachments 17 and 139 are assembled in a similar manner. Accordingly, the mounting of the attachments 17, 139 to handpiece 11 will only be described briefly herebelow.

With reference to FIGS. 1-3 and 5, to assemble the attachment 17 to the handpiece 11, cutting accessory 22 is first inserted through attachment 17 until shoulder 126 abuts the rearwardly facing surface of flange 61 of rear bearing sleeve 58, so that the accessory 22 is loosely fitted within attachment 17. An actuating collar 150 of handpiece 11 is rotated to the “load” position. The attachment 17 is then seated over the head end 18 of handpiece 11, and specifically over drill housings 151 and 152 thereof, so that the butt end 115 of accessory 22 inserted into a preload plunger 152A of handpiece 11 and into the drill housings 151 and 152. The attachment 17 is positioned over rear drill housing 151 so that the teeth 36 of retaining ring 33 are engaged within corresponding slots 153 of rear drill housing 151. With attachment housing 19 in this position, locking tabs 154 of a leaf spring 155 (which seats over rear drill housing 151) seat over two opposed lock ring teeth 36 of retainer ring 33 and lock the attachment housing 19 in position relative to the handpiece 11.

It will be appreciated that in the alternative, cutting accessory 22 can first be inserted into the drill housings 151 and 152 of handpiece 11 (after the collar 150 is rotated to the “load” position), and the attachment 17 then assembled to handpiece 11 over the accessory 22 and the head end 18 of handpiece 11.

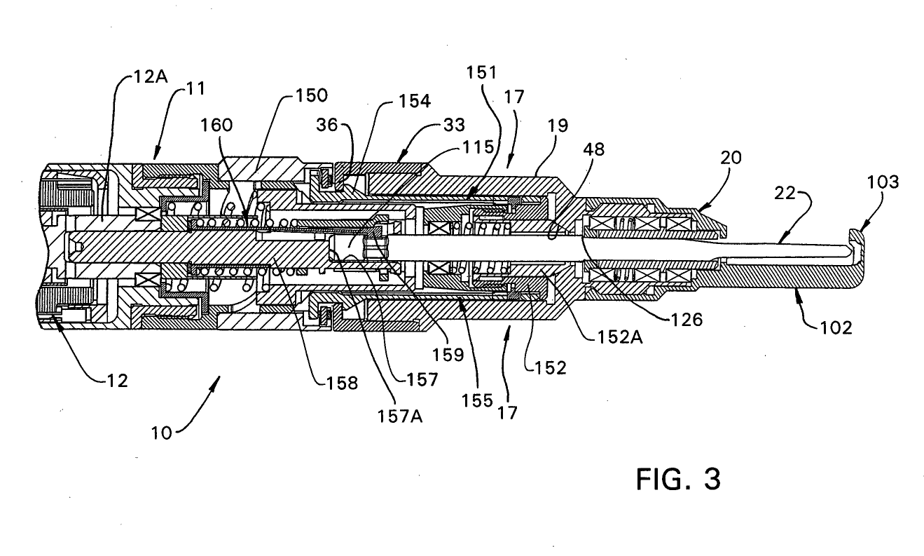

The actuating collar 150 of handpiece 11 is then rotated to the “run” position. In this regard, the handpiece 11 includes a collet housing 158 which is coupled to the rotor 12A for rotation therewith and includes a collet 160 which couples to the cutting accessory to cause rotation thereof along with rotor 12A. Collet housing 158 defines therein an accessory-receiving space 157 and an axially fixed, distally-facing surface 157A which defines the proximal terminal end of space 157. In the “run” position, the butt end 115 of cutting accessory 22 is seated in space 157 of collet housing 158, and the accessory 22 is rotationally locked to the collet housing 158 by the action of a plurality of collet feet 159 (only one of which is shown in FIG. 3) of collet 160 bearing against three of the faces 119 formed in the shaft locking section 116. The collet 160, firmly couples the accessory 22 to the rotor 12A such that the motor-induced rotation of rotor 12A is transferred to the accessory 22 to rotate same.

While the collet 160 as discussed above can be utilized to firmly axially hold or lock a cutting accessory within handpiece 11 as disclosed in the '200 patent, the collet 160 in accordance with the invention is only utilized to transfer rotational motion of rotor 12A to the cutting accessory, since the cooperation between the shoulder 126 of accessory 22 and the attachment 17 as discussed below serves to axially lock the accessory 12 within handpiece 11.

Attachment 17 with cap 20 as described above may be used to remove a section of the skull 162, as shown in FIG. 14. More specifically, first a bore is formed in the skull of the patient of a size sufficient to allow passage of the arm 102 and foot 103 into the skull 162. The handpiece 11 is then energized to rotate the cutting accessory 22, and the handpiece 11 is moved transversely along the skull 162 to cut with side or lateral cutting edges 132. During cutting, the surgeon normally applies upward pressure to the handpiece 11 so that the foot 103 is moved along and is positioned closely adjacent the inwardly directed side of the skull 162, which effectively peels or separates the dura 163 away from the skull 162. The foot 103 is located above or outside the dura 163 and protects same from damage by the tip 131 of the cutting accessory 22. The rounded outer surface of foot 103 also helps to prevent damage or trauma to the dura.

During cutting, it is of great importance that the tip 131 of the accessory 22 be correctly axially positioned within the pocket 108 defined in foot 103. For example, if during cutting the tip 131 bottoms out in the pocket 108, this could cause binding of the accessory 22 or other damage to the arrangement. Further, if the tip 131 is positioned too far from or out of the pocket 108 of foot 103, then the tip 131 will likely not cut through the entire thickness of the skull 162. The attachment 17 according to the invention prevents or at least significantly minimizes the possibility that any of the above will occur as discussed below.

When the attachment 17 is not installed on the handpiece 11 as shown in FIG. 4, the spring 81 exerts a distally directed force on second bearing 79, causing bearing 79 to exert a distally directed force on spacer 72 of front bearing sleeve 66, which in turn causes spacer 72 to exert a distally directed force on third bearing 84 and abut same against shoulder 101 of cap 20. At the same time, spring 81 exerts a proximally directed force on first bearing 75 to abut same against shoulder 53 of base 26, and positions rear bearing sleeve 58 a short axial distance forwardly from forwardly facing surface 50 of base 26. In this unassembled configuration of the handpiece 11 and attachment 17, the rear bearing sleeve 58 and the proximal end of the front bearing sleeve 66 are axially spaced from one another to define a gap G therebetween.

When the attachment 17 and accessory 22 are installed on the handpiece 11 as described above and as shown in FIGS. 3 and 5, the axially fixed surface 157A of collet housing 157 serves as a back-stop for the butt end 115 of accessory 22, and thus forces cutting accessory 22 forwardly a short distance, which in turn causes the shoulder 126 thereof to force rear bearing sleeve 58 and bearing 75 forwardly, which in turn compresses spring 81 against second bearing 79. This abutment of shoulder 126 against rear bearing sleeve 58 substantially eliminates the gap G between front and rear bearing sleeves 66 and 58 as shown in FIG. 5, and advances the cutting accessory 22 and thus the tip 131 thereof forwardly into the correct axial position relative to foot 103 of attachment 17, wherein the tip 131 is positioned within the pocket 108 of foot 103, but is not in contact with any inner surfaces of foot 103. Maintaining the tip 131 in this axial position relative to the attachment 17 effectively covers the distal-most end of tip 131 and cutting edges 133 thereof to protect the dura 163, so that only the lateral or side cutting edges 132 of accessory 22 are used.

The configuration of attachment 17 and shoulder 126 of accessory 22 as described above automatically positions the cutting accessory 22 in the optimum predetermined axial location relative to the foot 103 when the attachment 17 is secured to handpiece 11 by rearwardly biasing the butt end 115 of accessory 22 against the axially-fixed collet housing surface 157A of handpiece 11. Once the cutting accessory 22 and attachment 17 are installed on handpiece 11, as is shown in FIG. 5, a slight gap exists between the front and rear bearing sleeves 66 and 58. This gap is provided to compensate for manufacturing tolerances. However, if for some reason the cutting accessory 22 is pushed axially forwardly from its position shown on FIG. 5, the rear bearing sleeve 58 will axially abut the proximal face of front bearing sleeve 66. Since front bearing sleeve 66 is biased forwardly and thus is axially fixed by spring 81 relative to surface 101, further forward advancement of the cutting accessory 22 is prevented. Thus, the axial abutment of bearing sleeves 58 and 66 will prevent significant forward advancement of cutting accessory 22 relative to foot 103. The above configuration of attachment 17 also positions the cutting accessory 22 at the optimum rearward position within the pocket 108 of foot 103. That is, if the tip 131 of cutting accessory 22 is positioned out of or spaced to far rearwardly of pocket 108, the cutting accessory 22 will not cut through the entire transverse thickness of the skull 162.

Another aspect of the improved attachment according to the invention is that the front and rear bearing sleeves 66 and 58 are positioned radially between the cutting accessory 22 and the bearings 75, 79 and 84. One known arrangement, such as that disclosed in the '200 patent, allows for direct contact between the bearings and the rotating cutting accessory or shaft, which allows saline solution and other surgical debris to contaminate the bearings. The sleeves 66 and 58 according to the invention effectively protect the bearings 75, 79 and 84 from surgical debris, and route fluid through the attachment 17 and away from the bearings 75, 79 and 84, resulting in longer-life bearings.

To change or replace the attachment 17, such as with drill attachment 139 (for example, when a bore must be formed in the skull 162, such as prior to use of attachment 17 as discussed above), actuating collar 150 is rotated in the reverse direction from the “run” position past the “load” position and into the “eject” position, which forces the leaf spring locking tabs 154 inwardly, and the attachment 17 is forced in a direction away from the handpiece 11 through the action of the preload plunger 152A bearing against the inner surface of attachment base 26 which defines bore 48. The attachment 17 is removed from the handpiece 11, and actuating collar 150 is automatically returned to the “load” position (as described in the '200 patent). Attachment 139 is then secured into position on rear drill housing 151 of handpiece 11 in the same manner as attachment 17 as discussed above. If the same accessory 22 is to be used with attachment 139, then the accessory 22 can remain in the handpiece 11 while the attachment 17 is removed and the attachment 139 is installed on the handpiece 11.

Attachment 139 is typically used when cutting accessory 22 is to be utilized as a drill, wherein the end cutting surfaces or edges 133 of cutting accessory 22 are utilized to bore an opening into bone. As mentioned above, the same cutting accessory 22 is usable with both attachments 17 and 139, wherein the attachment 17 with its arm 102 and foot 103 effectively prevents usage of the end cutting surfaces 133, while the attachment 139 allows use of both cutting edges 133 and 132 of cutting accessory 22.

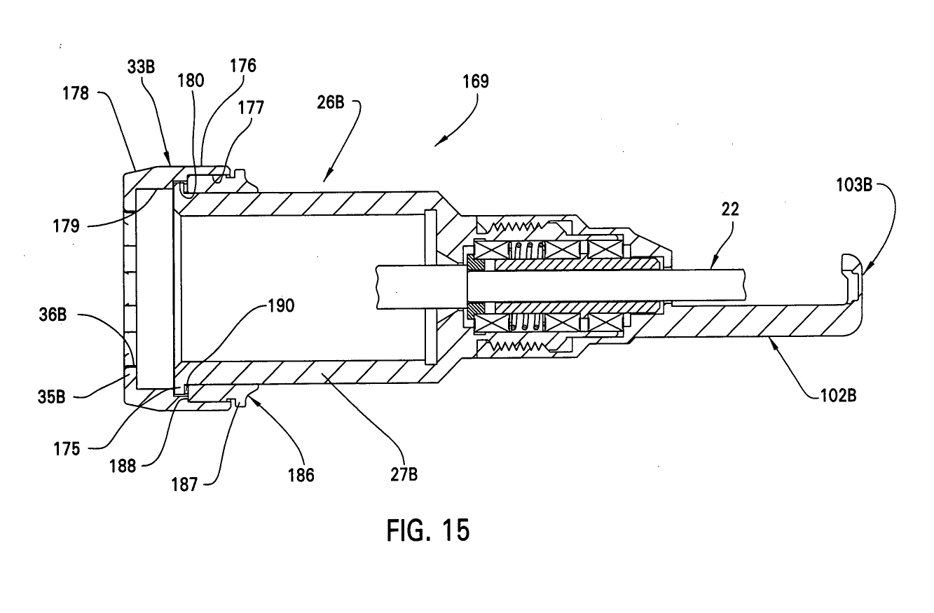

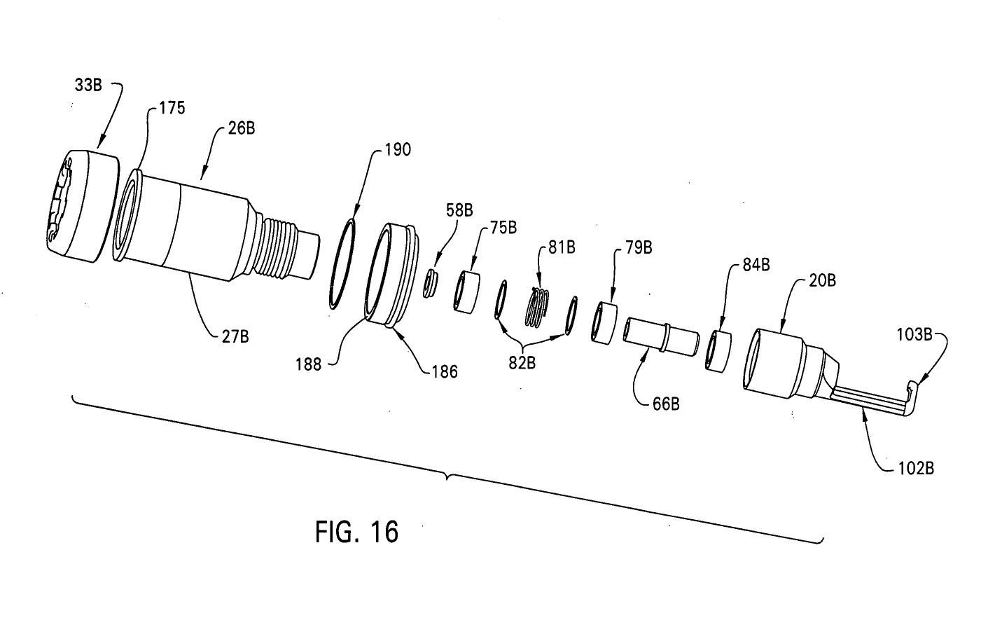

FIGS. 15 and 16 illustrate an additional attachment 169 which may be utilized with handpiece 11 according to the invention. Attachment 169 is similar to attachment 17, and similar or identical components will accordingly be referenced with the same reference numbers, plus a “B”.

Attachment 169 includes a cylindrical base 26B which is similar to base 26 of attachment 17, with the exception of its proximal end configuration. More specifically, base 26B is defined by a cylindrical part 27B which has a substantially constant outer diameter along the longitudinal extent thereof. An annular flange or lip 175 is cantilevered outwardly from the most proximal end of cylindrical part 27B, and is oriented generally perpendicularly relative to the outer surface thereof.

A retainer ring 33B is disposed at the proximal end of base 26B. Retainer ring 33B has a distally-located and sleeve-shaped terminal end 176 with an inner surface 177 defining a bore with a diameter greater than the outer diameter of cylindrical part 27B, and a proximally-located sleeve-shaped terminal end 178 joined to end 176. Sleeve-shaped end 178 defines stepped inner surfaces 179 and 180, wherein surface 180 defines a bore of a diameter greater than a bore defined by surface 179, but less than the diameter of surface 177. Further, terminal end 178 includes an inwardly projecting annular lip 35B defining a plurality of inwardly projecting teeth 36B which cooperate with the distal end of handpiece 11 to lock attachment 169 thereto.

An annular collar 186 is disposed in surrounding relation with the cylindrical part 27B adjacent flange 175. Collar 186 defines thereon an outwardly projecting and annular rib 187 at the distal end thereof, and a proximally-facing surface 188 at the opposite end thereof.

Retainer ring 33B is attached to base 27B, with the collar 186 interposed therebetween. More specifically, collar 186 is press-fitted into the bore defined by surface 177 of end 176 so as to be fixed to ring 33B, and ring 33B and collar 186 are slip-fitted onto base 27B. Flange 175 of base 27B is seated within the bore defined by proximal surface 180 of ring 33B with some radial clearance. An annular washer 190 is disposed between end surface 188 of collar 186 and a distally-facing surface of flange 175 of sleeve 27B. In the illustrated embodiment, washer 190 is a teflon washer.

In this embodiment, the base 27B is rotationally movable or pivotable relative to retainer ring 33B, collar 186 and handpiece 11, which allows the surgeon to “steer” the base 27B during surgery. The washer 190 reduces the rotational drag while providing some frictional resistance. More specifically, the surgeon can hold the handpiece 11 in one hand and use the fingers of the opposite hand to pivot or rotate the base 27B (and thus the foot 103B) relative to the ring 33B, collar 186 and handpiece 11. The attachment 169 is used in a manner similar to the attachment 17, and is attached and detached from the handpiece 11 in manner similar to attachment 17.

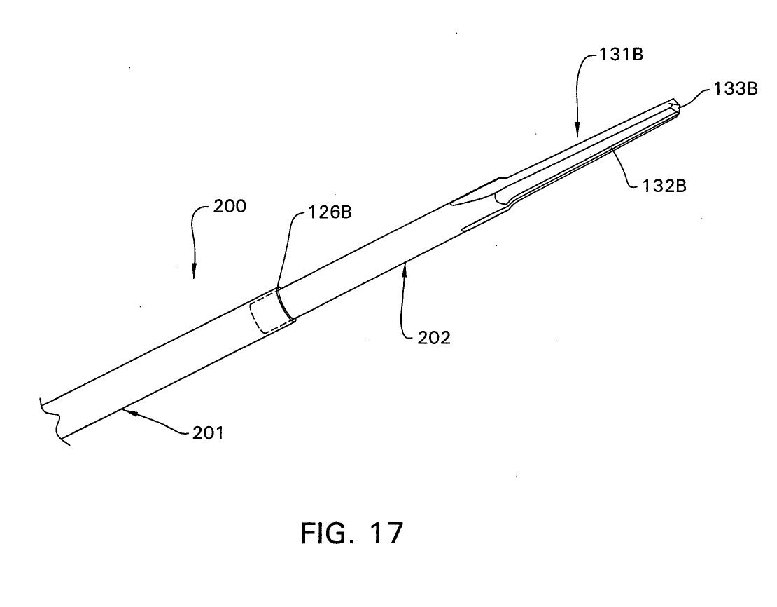

FIGS. 17-19 illustrate additional embodiments of cutting accessories which may be utilized in accordance with the invention. The cutting accessories shown in these figures are essentially identical to cutting accessory 22 described above, but show additional examples of how a shoulder or abutment may be formed on an intermediate part of the accessory for cooperation with attachments 17, 139 and 169. Accordingly, similar or identical components will accordingly be referenced with the same reference numbers, plus a “B”. It will be appreciated that the cutting accessories shown in FIGS. 17-19 all include a shaft locking section at their proximal end portion identical to shaft locking section 116 of accessory 22, although same are not shown for purposes of simplicity.

Cutting accessory 200 shown in FIG. 17 is initially a two-part assembly, including an elongated proximal and solid cylindrical part 201 which extends distally from the locking section (not shown), and an elongated distal and solid cylindrical part 202 extending forwardly from proximal part 201. Parts 201 and 202 are press-fit to one another, for example by inserting the proximal end of part 202 into a suitably-sized opening defined at the distal end of part 201. The proximal and distal parts 201 and 202 are of differing diameters. That is, distal part 202 is of a lesser diameter than proximal part 201, so that when the parts are joined, a shoulder or abutment 126B is formed, which is defined by the distal-most terminal end of proximal part 201. Alternatively, parts 201 and 202 can joined by welding, or by providing a threaded end on one part and a threaded opening on the other part.

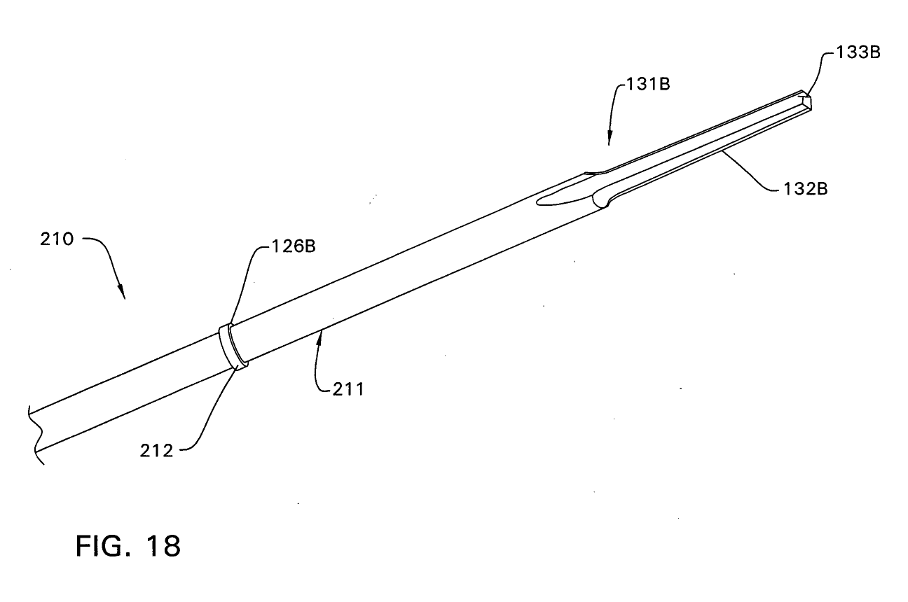

Cutting accessory 210 shown in FIG. 18 includes an elongated and solid cylindrical portion 211 which extends proximally from cutting tip 131B. Cylindrical portion 211 has a constant diameter along the length thereof. A ring 212 is press-fit onto the outer diameter of the cylindrical portion 201 to a set height. A forwardly facing surface of the ring 212 defines a shoulder 126B.

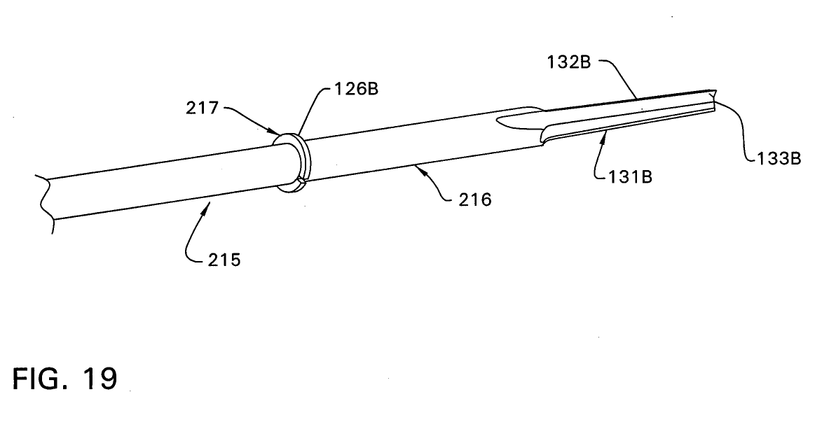

Cutting accessory 215 shown in FIG. 19 includes an elongated and solid cylindrical portion 216 which extends proximally from cutting tip 131B. Portion 216 has a constant diameter except at an area intermediate the ends of accessory 215, wherein an annular and shallow groove is defined about the circumference of portion 216. A spring-like retaining ring 217 is positioned within the groove, and a forwardly facing surface thereof defines a shoulder 126B.

It will be appreciated the shoulders 126, 126B defined on the cutting accessories described above all include an axially unobstructed, forwardly or distally facing surface which projects radially beyond the diameter of the portion of the cutting accessory defined on the distal side of the respective shoulder (i.e. parts 123 and 202 of FIGS. 9 and 17), or alternatively beyond both portions of the cutting accessory located on both the proximal and distal sides of the respective shoulder (FIGS. 18 and 19).

Although a particular preferred embodiment of the invention is disclosed in detail for illustrative purposes, it will be recognized that variations or modifications of the disclosed apparatus, including the rearrangement of parts, lie within the scope of the present invention.

What is claimed is:

1. A combination attachment for removable connection to a surgical handpiece and a cutting accessory, said combination comprising:
a cutting accessory comprising an elongate shaft defining a longitudinal axis and including a proximal end portion for disposal adjacent a handpiece and a distal end portion defining at least one cutting edge thereon, said shaft defining thereon a surface extending generally transversely relative to the longitudinal axis and projecting radially beyond an outer diameter of a portion of said cutting accessory;
an attachment including a housing having one end configured for connection to a surgical handpiece and defining a generally hollow interior for accommodating said cutting accessory therein, said attachment including a positioning arrangement disposed within said hollow interior and defining a surface extending generally transversely relative to the longitudinal axis of the shaft and which is disposed adjacent to and cooperates with said shaft surface to maintain said shaft in a predetermined axial position relative to said attachment when said attachment is coupled to a handpiece.

2. The combination of claim 1, wherein said one end is a first end and said attachment includes a second end spaced from said first end and defining thereon a foot which is oriented generally transversely relative to the longitudinal axis and extends over said distal end portion of said shaft, said positioning arrangement maintaining said shaft in said predetermined axial position to prevent engagement of said distal end portion of said shaft with said foot.

3. The combination of claim 1, wherein said positioning arrangement includes a bearing sleeve defining said surface thereon, said bearing sleeve being disposed in surrounding relation with said shaft.

4. The combination of claim 1, wherein said positioning arrangement includes a biasing member disposed to urge said shaft in a direction towards a handpiece when said attachment is installed thereon.

5. The combination of claim 3, wherein said positioning arrangement includes a biasing member disposed to urge said bearing sleeve and said shaft in a direction towards a handpiece when said attachment is installed thereon.

6. The attachment of claim 3, wherein said bearing sleeve is a first bearing sleeve and said positioning arrangement includes a second bearing sleeve disposed in surrounding relation with said shaft in coaxial relation with said first bearing sleeve, and a second bearing sleeve being disposed a further distance from said one end of said attachment than said first bearing sleeve, and a biasing member disposed to bias said first bearing sleeve and said shaft in a first direction towards said one end of said attachment and a handpiece and to bias said second bearing sleeve in a second direction opposite said first direction.

7. The attachment of claim 6, wherein at least one annular ball bearing is disposed in surrounding relation with said first and second bearing sleeves within said hollow interior radially between said housing and said shaft.

8. The attachment of claim 6, wherein said biasing member is disposed to bias said second bearing sleeve and maintain same in a fixed axial position relative to said housing, and said first bearing sleeve and said shaft are movable relative to said second bearing sleeve in said second direction against the biasing force of said biasing member during connection of said attachment to a handpiece, to automatically position said shaft in a predetermined axial position relative to said housing.

9. A combination attachment and cutting accessory for use with a surgical handpiece, said combination comprising:
a cutting accessory including an elongate shaft defining a longitudinal axis and having a first proximal end for disposal adjacent a handpiece and a second distal end defining a cutting edge thereon, said shaft defining thereon a shoulder disposed between said first and second ends; and
an attachment including a housing having a first proximal end configured for securing said attachment to a handpiece and a second distal end spaced from said first end, said housing defining a through-bore therein wherein said shaft extends through said bore and has a portion which projects outwardly beyond said second end of said housing, said second end of said housing mounting thereon an arm disposed adjacent said shaft portion and defining a terminal end portion oriented transversely relative to the longitudinal axis, said attachment further including a positioning member disposed to exert a biasing force on said shoulder to urge said shaft into engagement with an axially fixed surface of a handpiece to maintain said second end of said shaft at a predetermined axial distance from said terminal end portion of said arm.

10. The combination of claim 9, wherein said positioning member comprises an annular bearing member disposed in surrounding relation with said shaft and engaging said shoulder to position said shaft in abutting relation with an axially fixed surface of a handpiece.

11. The combination of claim 9, wherein said terminal end portion comprises a foot defining a pocket therein which opens proximally, said positioning member positioning said second end of said shaft within said pocket for movement relative to said terminal end portion.

12. The combination of claim 10, wherein said bearing member is a first bearing member and said attachment includes a second sleeve-like bearing member disposed in surrounding relation with said shaft axially adjacent said first bearing member, and a biasing member disposed to urge said first bearing member in a first direction towards said first end of said attachment and said second bearing member in a second direction opposite said first direction and into abutment with a fixed surface defined at said second end of said housing, said first and second bearing members being movable into axially abutting relationship with one another when said attachment and said accessory are secured to a handpiece to prevent advancement of said shaft in said second direction.

13. The combination of claim 12, wherein said attachment includes at least one annular ball bearing disposed radially between said first and second bearing members and said housing.

14. An attachment for connection to and use with a powered surgical handpiece and for receiving therein a rotating surgical cutting accessory, said attachment comprising:
a housing defining a longitudinal axis and a generally hollow interior for receiving therein a rotating surgical cutting accessory, a first proximal end for disposal adjacent a handpiece and a second distal end spaced from said first end; and
a bearing arrangement for supporting the cutting accessory for rotation relative thereto, said bearing arrangement including at least one annular ball bearing disposed in surrounding relation with a cutting accessory closely adjacent said second end of said housing, and a sleeve arrangement disposed in surrounding relation with a cutting accessory radially between said ball bearing and a cutting accessory to prevent contamination of said ball bearing by surgical debris entering said attachment, said sleeve arrangement being disposed to cooperate with a cutting accessory to maintain same in a predefined axial position relative to said attachment.

15. The attachment of claim 14, wherein said sleeve arrangement includes a pair of annular sleeves disposed in axially adjacent relationship with one another in surrounding relation with a cutting accessory.

16. The attachment of claim 15, wherein said sleeve arrangement includes a first sleeve disposed adjacent said proximal end of said housing and a second sleeve disposed adjacent said distal end thereof, a first ball bearing disposed radially between said housing and said first and second sleeves, a second ball bearing disposed radially between said housing and a distal end of said second sleeve, said first and second ball bearings being disposed to support a cutting accessory for rotation within and relative to said attachment, and a biasing member disposed between said first and second ball bearings and urging said first bearing and said first sleeve in a first direction towards said first proximal end of said housing and said second bearing and said second sleeve in a second direction opposite said first direction.

17. An attachment for connection to and use with a powered surgical handpiece and for receiving therein a rotating surgical cutting accessory, said attachment comprising a housing defining a longitudinal axis and a generally hollow interior for receiving therein a rotating surgical cutting accessory, a first end for connection to a powered surgical handpiece and a second end remote from said first end, a biasing arrangement disposed within said housing for resiliently urging a surgical cutting accessory in a direction away from said second end and into engagement with an axially fixed surface of a powered surgical handpiece.

18. A combination attachment for connection to and use with a powered surgical handpiece and a surgical cutting accessory, said combination comprising:
a cutting accessory including an elongated shaft defining a longitudinal axis and having a first end defining at least one cutting edge thereon and a second end remote from said first end; and
an attachment including a housing having a first end associated with a powered surgical handpiece and a second end remote from said attachment first end, said housing receiving therein said shaft of said cutting accessory, and a biasing member disposed within said housing and engaging said shaft to urge same in a direction generally parallel to the longitudinal axis away from said attachment second end and into engagement with an axially fixed surface of a powered surgical handpiece.

19. The combination of claim 18, wherein said cutting accessory defines thereon an abutment disposed adjacent said biasing member, said biasing member engaging said abutment to urge said second end of said cutting accessory into engagement with an axially fixed surface of a powered surgical handpiece.

20. The combination of claim 19, wherein said abutment defines thereon a face which faces towards said first end of said cutting accessory, said face being axially unobstructed by a remainder of said shaft.

21. The combination of claim 19, wherein said abutment projects radially beyond an outer diameter of said shaft.

22. A surgical tool arrangement comprising:
a cutting accessory defining a longitudinal axis and having a first proximal end, a second distal end spaced from said first end and defining at least one cutting edge thereon, and an abutment disposed between said first and second ends;
a handpiece having a first proximal end, a second distal end, a housing defining a longitudinal axis, and a motor having a rotor element; and
an attachment having a first proximal end for coupling with said second end of said housing and a second distal end, and a housing defining a generally hollow interior, said second end of said cutting accessory being disposed in said interior of said attachment and said cutting accessory being rotatable with said rotor element, said attachment including a positioning member within said interior and disposed to cooperate with said shaft abutment to automatically position said cutting accessory in a predetermined axial position relative to said attachment upon coupling of said attachment to said second end of said handpiece.

23. The tool arrangement of claim 22; wherein said positioning member comprises an annular member disposed in surrounding relation with said cutting accessory, said annular member defining a surface thereon which engages with said shaft abutment.

24. The tool arrangement of claim 23, wherein said attachment includes a biasing element which exerts a biasing force on said cutting accessory through said annular member to urge said cutting accessory in a direction towards said handpiece and into engagement with said rotor member thereof.

25. The tool arrangement of claim 22, wherein said positioning member comprises a biasing element which is disposed to bias said cutting accessory in a direction towards said handpiece and into engagement with an axially fixed surface of said rotor member.

26. The tool arrangement of claim 22, wherein said cutting accessory is defined by an elongate shaft having a cutting tip defined at said second end of said accessory, said second end of said attachment defining thereon a foot which is oriented generally transversely relative to the longitudinal axis of said cutting accessory and extends over said cutting tip thereof, said positioning member maintaining said shaft in said predetermined axial position to prevent engagement of said cutting tip with said foot.

27. The tool arrangement of claim 22, wherein said positioning member comprises a first annular bearing element disposed in surrounding relation with said cutting accessory and in engagement with said shaft abutment, and a second annular bearing element is disposed in surrounding relation with said cutting accessory in coaxial relation with said first bearing element, said second bearing element being disposed a further distance from said first end of said attachment than said first bearing element, and a biasing member is disposed to bias said first bearing element and said cutting accessory in a first direction towards said first end of said attachment and said handpiece and to bias said second bearing element in a second direction opposite said first direction into engagement with a fixed surface of said attachment housing.

28. The tool arrangement of claim 27, wherein said attachment includes at least one annular ball bearing disposed in surrounding relation with said first and second bearing elements within said attachment interior.

29. The tool arrangement of claim 28, wherein said biasing member is disposed to bias said second bearing element and maintain same in a fixed axial position relative to said surface of said attachment housing, and said first bearing element and said cutting accessory are movable relative to said second bearing element in said second direction against the biasing force of said biasing member during connection of said attachment to said handpiece, to automatically position said cutting accessory in said predetermined axial position relative to said attachment housing.

30. The tool arrangement of claim 29, wherein connection of said attachment to said handpiece causes said first bearing element to normally move into closely adjacent relation with said second bearing element, said first and second bearing elements being movable into abutting engagement with one another upon advancement of said cutting accessory in said second direction, and the engagement of said second bearing element with said fixed surface of said attachment prevents further movement of said first bearing element and said cutting accessory in said second direction.

31. A surgical tool arrangement comprising:
a cutting accessory having a shaft defining a longitudinal axis, a first end and a second end remote from said first end and defining a cutting edge thereon;
a handpiece having a housing and a rotating element disposed in said housing and associated with a handpiece motor, said rotating element being rotatably coupled to said cutting accessory; and
an attachment having one end coupled with said handpiece housing, a generally hollow housing in which said cutting accessory is disposed, and a biasing member disposed within said attachment housing and engaging said shaft to urge said shaft in a direction generally parallel to the longitudinal axis towards said handpiece and into engagement with an axially fixed portion of said rotating element.

32. The tool arrangement of claim 31, wherein said biasing member and said portion of said rotating element maintain said shaft in a predetermined axial position relative to said attachment.

33. The tool arrangement of claim 31, wherein said attachment has another end opposite said one end which defines thereon a foot oriented transversely relative to the longitudinal axis and extending over said second end of said cutting accessory, said biasing member and said portion of said rotating element maintaining said shaft in a predetermined axial position relative to said attachment to prevent engagement of said second end of said cutting accessory with said foot.
